(19) United States
(12) Patent Application Publication
(10) Pub. No.: US 2008/0255658 A1
(43) Pub. Date: Oct. 16, 2008

(54) Degradation Associated Drug Delivery for Drug Eluting Stent and Medical Device Coatings

(75) Inventors: Brian Cook, Windsor, CA (US); Joseph Berglund, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/734,388

(22) Filed: Apr. 12, 2007

(51) Int. Cl. A61F 2/06

(52) U.S. Cl. 623/1.42

(57) ABSTRACT

A system for treating a vascular condition includes a stent having a biodegradable coating on the stent framework. The coating releases a therapeutically effective amount of therapeutic agent as a function of degradation of the coating. Also provided is a method of treating a vascular condition by placing a stent having a biodegradable coating at a treatment site and delivering a therapeutically effective amount of therapeutic agent at the treatment site as a function of degradation of the coating. Also provided is a method of improving the performance of a medical device by including a biodegradable coating on the surface of the device and releasing a therapeutically effective amount of a therapeutic agent at the treatment site as a function of degradation of the coating.

### TECHNICAL FIELD

This invention relates generally to biomedical devices that are used for treating vascular conditions. More specifically, the invention relates to a therapeutic agent eluting stent having a biodegradable coating that releases one or more therapeutic agents as a function of degradation of the coating.

### BACKGROUND OF THE INVENTION

Stents are generally cylindrical-shaped devices that are radially expandable to hold open a segment of a vessel or other anatomical lumen after implantation into the body lumen.

Various types of stents are in use, including expandable and self-expanding stents. Expandable stents generally are conveyed to the area to be treated on balloon catheters or other expandable devices. For insertion into the body, the stent is positioned in a compressed configuration on the delivery device. For example, the stent may be crimped onto a balloon that is folded or otherwise wrapped about the distal portion of a catheter body that is part of the delivery device. After the stent is positioned across the lesion, it is expanded by the delivery device, causing the diameter of the stent to expand. For a self-expanding stent, commonly a sheath is retracted, allowing the stent to expand.

Stents are used in conjunction with balloon catheters in a variety of medical therapeutic applications, including intravascular angioplasty to treat a lesion such as plaque or thrombus. For example, a balloon catheter device is inflated during percutaneous transluminal coronary angioplasty (PTCA) to dilate a stenotic blood vessel. When inflated, the pressurized balloon exerts a compressive force on the lesion, thereby increasing the inner diameter of the affected vessel. The increased interior vessel diameter facilitates improved blood flow. Soon after the procedure, however, a significant proportion of treated vessels restenose.

To reduce restenosis, stents, constructed of metals or polymers, are implanted within the vessel to maintain lumen size. The stent is sufficiently longitudinally flexible so that it can be transported through the cardiovascular system. In addition, the stent requires sufficient radial strength to enable it to act as a scaffold and support the lumen wall in a circular, open configuration

Stent insertion may cause undesirable reactions such as inflammation resulting from a foreign body reaction, infection, thrombosis, and proliferation of cell growth that occludes the blood vessel. Stents capable of delivering one or more therapeutic agents have been used to treat the damaged vessel and reduce the incidence of deleterious conditions including thrombosis and restenosis.

Polymer coatings applied to the surface of the stents have been used to deliver drugs or other therapeutic agents at the placement site of the stent. The coating may comprise biodegradable or biostable polymers singly, or in various combinations to give the coating unique properties such as controlled rates of degradation, or a biostable mesh with a biodegradable or bioerodable portions that control elution of the therapeutic agent. However, some biostable polymers have been found to be irritating to the tissues they contact during long term implantation. In addition, some biodegradable polymers generate acidic byproducts and degradation products that elicit an inflammatory response.

It would be desirable, to provide an implantable therapeutic agent eluting stent having a biodegradable polymeric coating capable of releasing one or more therapeutic agents at a therapeutically efficacious rate and releasing one agent (an anti-inflammatory, for example) during the entire degradation phase of the coating. Such a stent would overcome many of the limitations and disadvantages inherent in the devices described above.

### SUMMARY OF THE INVENTION

One aspect of the present invention provides a system for treating a vascular condition comprising a catheter and a therapeutic agent-carrying stent disposed on the catheter. The stent includes a coating disposed on the surface of the stent, and at least one therapeutic agent contained within the coating. Following initial release of the therapeutic agent, the coating retains a sufficient amount of therapeutic agent to allow release of a therapeutically effective amount of the therapeutic agent as a function of degradation of the coating. Another aspect of the invention provides a stent having a coating on at least a portion of the surface of the stent. Following initial release of a therapeutic agent from the coating, the coating retains and releases a therapeutically effective amount of therapeutic agent as a function of degradation of the coating.

Another aspect of the invention provides a method for treating a vascular condition by delivering a stent having a biodegradable coating and at least one therapeutic agent to a treatment site via catheter. The method further comprises delivering a therapeutically effective amount of the therapeutic agent to the treatment site as a function of degradation of the coating.

Yet another aspect of the invention provides a method of improving the performance of a medical device by providing the medical device with a biodegradable coating disposed on the surface of the device. Included within the coating is at least one therapeutic agent. A therapeutically effective amount of the therapeutic agent is released at the treatment site at a predetermined rate as a function of degradation of the coating.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying drawings.

### BRIEF DESCRIPTION OF THE DRAWINGS

FIG. 1 is a schematic illustration of a system for treating a vascular condition comprising a therapeutic agent carrying stent coupled to a catheter, in accordance with one embodiment of the present invention;

FIG. 2A is a schematic illustration of a coating containing a therapeutic agent on the surface of a stent or other medical device;

FIG. 2B is a schematic illustration of a therapeutic agent being delivered to a treatment site from a coating on a stent of other medical device;

FIG. 3 is a schematic illustration of a lock-in phase of therapeutic agent delivery from a coating on a stent or other medical device, in accordance with the present invention;

FIG. 4 is a schematic illustration of release of a therapeutic agent from a coating on a stent or other medical device as a function of the degradation phase of the coating, in accordance with the present invention;

FIG. 5A is a graphical representation of the time course of zotarolimus release from poly-lactide-co-glycolide (50:50, molar ratio), in accordance with the present invention;

FIG. 5B is a graphical representation of the degradation profile for poly-lactide-co-glycolide (50:50, molar ratio);

FIG. 5C is a graphical representation of the mass loss due to polymer degradation and drug elution, in accordance with the present invention;

FIG. 6 is a flow diagram of a method for treating a vascular condition, in accordance with the present invention; and

FIG. 7 is a flow diagram of a method for improving the performance of a medical device, in accordance with the present invention.

### DETAILED DESCRIPTION

Throughout this specification, like numbers refer to like structures.

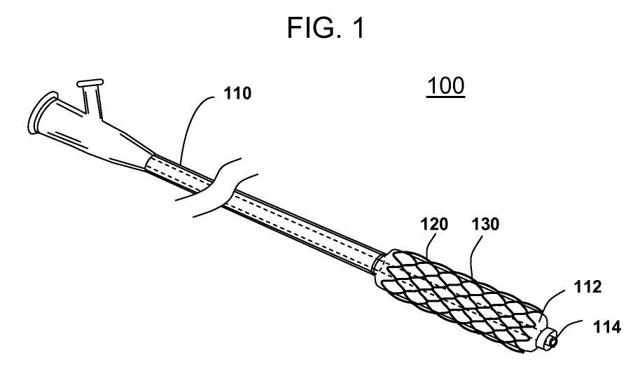

The present invention is directed to a system for treating abnormalities of the cardiovascular system comprising a catheter and a therapeutic agent-carrying stent disposed on the catheter. A biodegradable coating disposed on the surface of the stent retains a portion of the therapeutic agent. During the degradation phase of the coating, a therapeutically effective amount of the therapeutic agent is released at the treatment site. In an exemplary embodiment of the invention, FIG. 1 shows an illustration of a system 100 comprising therapeutic agent carrying stent 120 coupled to catheter 110. Catheter 110 includes a balloon 112 that expands and deploys therapeutic agent carrying stent 120 within a vessel of the body. After positioning therapeutic agent carrying stent 120 within the vessel with the assistance of a guide wire traversing through guide wire lumen 114 inside catheter 110, balloon 112 is inflated by pressurizing a fluid such as a contrast fluid or saline solution that fills a lumen inside catheter 110 and balloon 112. Therapeutic agent carrying stent 120 is expanded until a desired diameter is reached; then the contrast fluid is depressurized or pumped out, separating balloon 112 from therapeutic agent carrying stent 120 and leaving the therapeutic agent carrying stent 120 deployed in the vessel of the body. Alternately, catheter 110 may include a sheath that retracts to allow expansion of a self-expanding embodiment of therapeutic agent carrying stent 120. Therapeutic agent carrying stent 120 includes a stent framework 130. In one embodiment of the invention, a porous coating is disposed on the surface of at least a portion of stent framework 130.

In one embodiment of the invention, the stent framework comprises one or more of a variety of biocompatible metals such as stainless steel, titanium, magnesium, aluminum, chromium, cobalt, nickel, gold, iron, iridium, chromium/titanium alloys, chromium/nickel alloys, chromium/cobalt alloys, such as MP35N and L605, cobalt/titanium alloys, nickel/titanium alloys, such as nitinol, platinum, and platinum-tungsten alloys. The metal composition gives the stent framework the mechanical strength to support the lumen wall of the vessel, sufficient longitudinal flexibility so that it can be transported through the cardiovascular system, and provides a metallic substrate for the oxidation and reduction reactions that produce a porous coating.

In another embodiment of the invention, stent framework 130 comprises one or more biocompatible polymeric materials. Polymeric stent framework 130 may be biodegradable, biostable, or comprise a mixture of polymeric materials that are both biostable and biodegradable. Biodegradable polymers appropriate for the stents of the invention include polylactic acid, polyglycolic acid, and their copolymers, caproic acid, polyethylene glycol, polyanhydrides, polyacetates, polycaprolactones, poly(orthoesters), polyamides, polyurethanes and other suitable polymers. Biostable polymers appropriate for the stents of the invention include polyethylene, polypropylene, polymethyl methacrylate, polyesters, polyamides, polyurethanes, polytetrafluoroethylene (PTFE), polyvinyl alcohol, and other suitable polymers. These polymers may be used alone or in various combinations to give the stent unique properties such as controlled rates of degradation.

The stent framework is formed by shaping a metallic wire or polymeric filament, or by laser cutting the stent from a metallic or polymeric sheet, or any other appropriate method. If needed, the surface of the stent framework is cleaned by washing with surfactants to remove oils, mechanical polishing, electropolishing, etching with acid or base, or any other effective means to expose a clean, uniform surface that is ready for applying a coating.

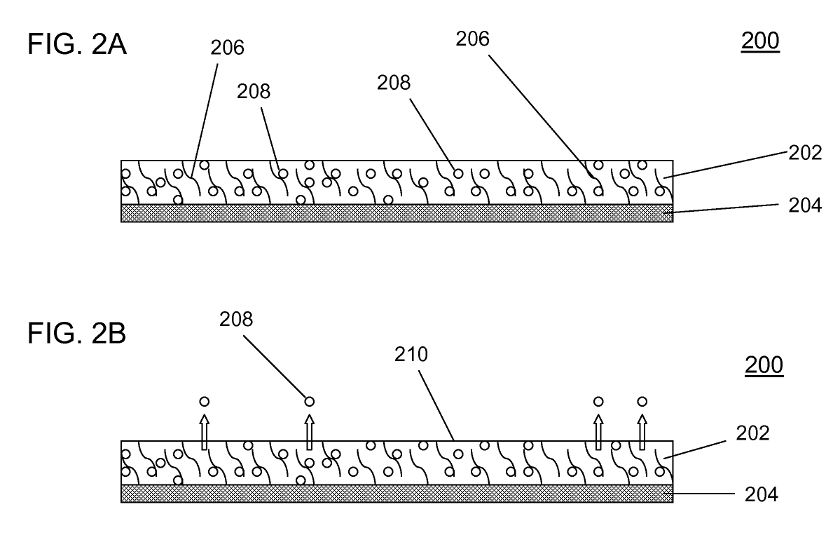

FIG. 2A is an illustration of a system 200 comprising therapeutic agent delivery coating 202 disposed on surface 204 of a stent framework or other medical device. Coating 202 includes polymer matrix 206 comprising biodegradable polymers 206 such as polylactic acid, polyglycolic acid, and their copolymers, caproic acid, polyethylene glycol, polyanhydrides, polyacetates, polycaprolactones, poly(orthoesters), polyamides, polyurethanes and other suitable polymers.

In one embodiment of the invention, therapeutic agent molecules 208 are contained within coating 202. Various therapeutic agents, such as anticoagulants, antiinflammatories, fibrinolytics, antiproliferatives, antibiotics, therapeutic proteins or peptides, recombinant DNA products, or other bioactive agents, diagnostic agents, radioactive isotopes, or radiopaque substances may be used depending on the anticipated needs of the targeted patient population. In one embodiment the therapeutic agent is selected from the group consisting of zotarolimus, everolimus, sirolimus, pimecrolimus, dexamethasone, hydrocortisone, salicylic acid, fluocinolone acetonide, corticosteroids, prodrugs thereof, and combinations thereof. The formulation containing the therapeutic agent may additionally contain excipients including solvents or other solubilizers, stabilizers, suspending agents, antioxidants, and preservatives, as needed to deliver an effective dose of the therapeutic agent to the treatment site. The polymeric coating containing therapeutic agent 208 may be applied to the surface 204 of stent framework 130 by any means known in the art such as, for example, by spraying or dipping stent framework 130.

Therapeutic agent molecules 208 are held within coating 202 by entrapment within the polymer mesh of the coating, or by chemical means such as hydrogen bonding, or hydrophobic interactions depending on the polarity and solubility of therapeutic agent molecules 208. After delivery to the treatment site, therapeutic agent molecules 208 near surface 210 are rapidly released from coating 202 by diffusing out through surface 210 (FIG. 2B). This phenomenon causes an initial burst of therapeutic agent release. As molecules 208 near surface 210 of coating 202 are depleted, a concentration gradient develops across the thickness of coating 202 in which there is a greater concentration of molecules 208 near stent surface 204. This concentration gradient causes molecules 208 to continually migrate toward surface 210 and diffuse out of coating 202 at surface 210 until the concentration of molecules 208 remaining in coating 202 is too low to support gradient formation. This second phase of therapeutic agent release follows a release-kinetics profile such as zero order release (linear), or first order release (hyberbolic), depending on the physical and chemical interactions between therapeutic agent molecules 208 and polymer matrix 206.

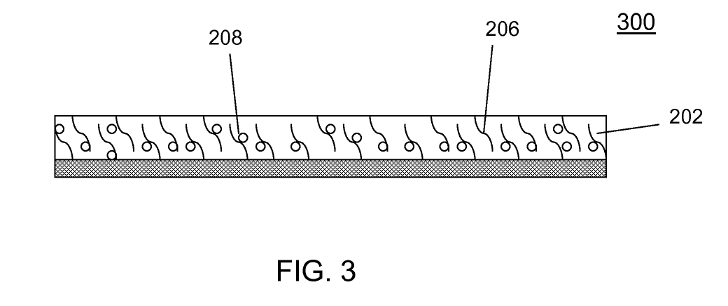

Shown in FIG. 3, as therapeutic agent molecules 208 continue to migrate out of coating 202, the concentration of therapeutic agent molecules 208 remaining within coating 202 becomes too low to form a sufficiently steep gradient to allow migration of therapeutic agent molecules 208 out of coating 202 to continue. However some therapeutic agent molecules 208 remain entrapped within coating 202. This is referred to as lock-in phase 300 of therapeutic agent delivery.

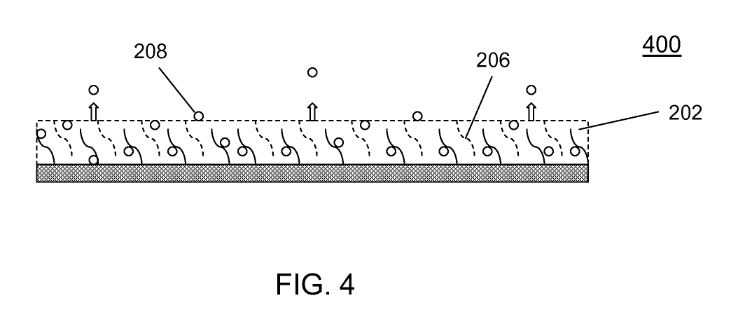

If coating 202 comprises polymers that are biodegradable under physiological conditions, coating 202 begins to degrade soon after placement of the stent or other medical device at the treatment site. As polymer matrix 206 of coating 202 degrades, therapeutic agent molecules 208 that were locked in coating 202 are released, as shown in FIG. 4. In this degradation-associated phase 400 of therapeutic agent release, the amount of therapeutic agent 208 released is determined by the rate of breakdown of coating 202 and the amount of therapeutic agent 208 remaining within the coating matrix in lock-in phase 300. In one embodiment of the invention, polymers 206 comprising the matrix of coating 202 are selected to entrap sufficient therapeutic agent 208 in lock-in phase 300 to provide a therapeutically effective amount of agent at the treatment site resulting from release of the therapeutic agent during degradation of coating 202 in degradation-associated phase 400 of drug release. In one embodiment, degradation-associated therapeutic agent release 400 takes place over a period of three to six months.

In one embodiment of the invention, two therapeutic agents are contained within biodegradable coating 202. A first therapeutic agent is released by migration out of the coating and the second therapeutic agent remains locked in the coating. As coating 202 breaks down, a therapeutically effective amount of the second therapeutic agent is released. In one embodiment, sirolimus is released as the first agent, and dexamethasone is released as the second agent. In another embodiment, zotarolimus is released as the first agent, and fluocinolone acetonide is released as the second agent.

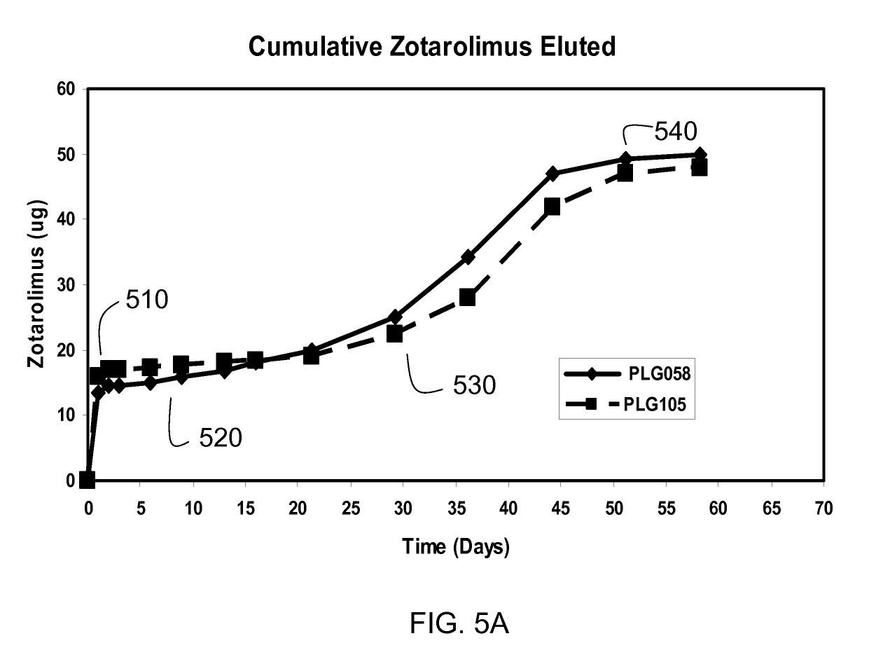

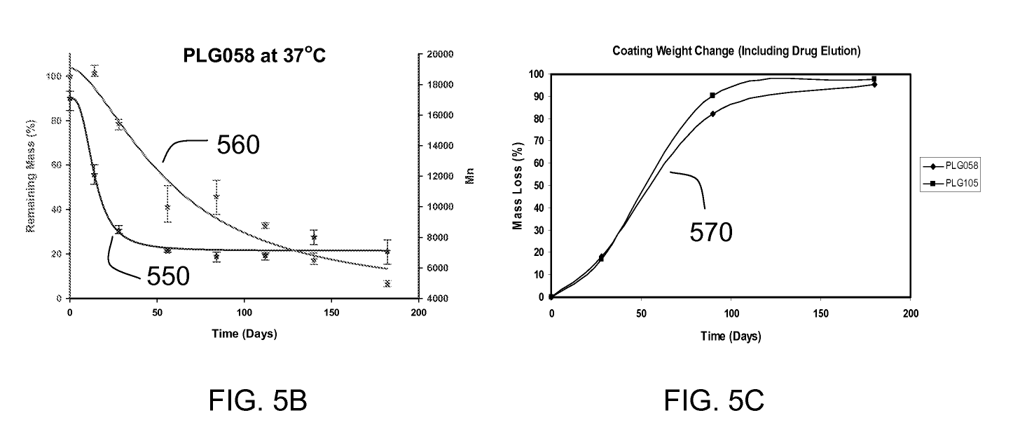

In another embodiment of the invention polymer matrix 206 of coating 202 comprises poly-lactide-co-glycolide (50:50 molar ratio) polymers. Therapeutic agent 208 is sirolimus or zotarolimus. In one embodiment therapeutic agent 208 is zotarolimus at a concentration between 10 and 35% weight/weight of coating 202. Release of 25% zotarolimus in this embodiment is portrayed in FIG. 5A. PLG058 refers to a polymer with a 0.58 ml/g inherent viscosity while PLG105 refers to a polymer with a 1.05 ml/g inherent viscosity when tested in chloroform at 25° C. Following placement of the stent, there is an initial burst of zotarolimus release 510 for approximately one day. Next, therapeutic agent lock-in 520 occurs, and little additional zotarolimus is released for approximately twenty days. Then degradation associated release 530 begins and continues until the polymer has substantially degraded and the therapeutic agent is fully exhausted 540. Presented in FIG. 5B are degradation profiles for poly-lactide-co-glycolide (50:50 molar ratio) showing molecular weight decrease 550 and coating mass loss 560 during the degradation associated release of zotarolimus, shown between points 530 and 540 in FIG. 5A. Similar coating weight loss degradation profiles 570 are observed for stents coated with 25% zotarolimus and are shown in FIG. 5C. In this embodiment, the amount of zotarolimus released during the degradation associated release phase is sufficient to reduce inflammation at the treatment site.

In another embodiment, paclitaxel is released from a coating comprising caprolactone and glycolide polymers, in which glycolide comprises between 50 and 99% of the polymer matrix. In another embodiment, zotarolimus is released from a coating containing trimethylene carbonate (TMC), lactide, and glycolide polymers, in which the cumulative amount of lactide and glycolide comprises between 50 and 99% of the polymer matrix.

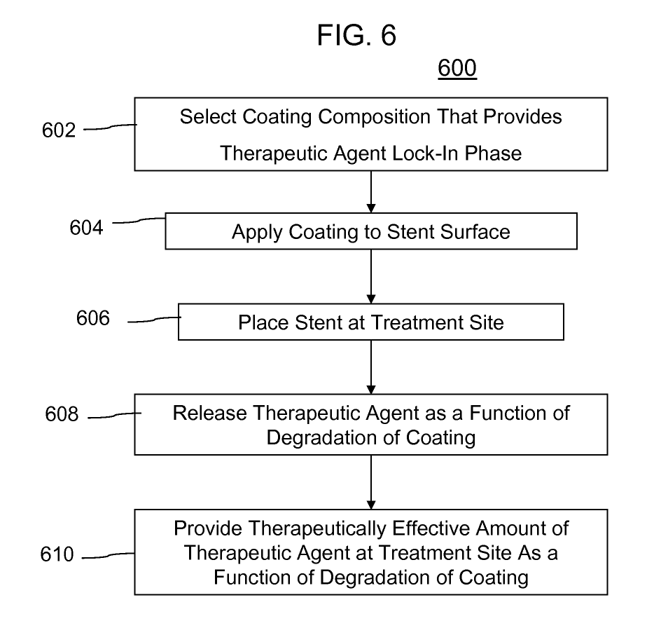

FIG. 6 is a flowchart of method 600 for treating a vascular condition using a therapeutic agent eluting stent, in accordance with the present invention. The method includes selecting a coating for a stent that will hold a sufficient amount of therapeutic agent locked within the coating matrix to provide a therapeutically effective amount of therapeutic agent during degradation associated release, as indicated in Block 602. The coating comprises biodegradable polymers, one or more therapeutic agents to be delivered, and any other excipients needed to cause the coating to adhere to the surface of the stent framework, and deliver the therapeutic agent(s) to the treatment site. As indicated in Block 604 the coating is applied to the surface of the stent frame work. In one embodiment of the invention, the coating is applied as a liquid by dipping or spraying, and then dried to remove solvent using air, vacuum, or heat, and any other effective means of causing the formulation to adhere to the stent framework.

Next, as indicated in Block 606, the coated, therapeutic agent eluting stent is mounted on a catheter and delivered to the treatment site. At the treatment site, the stent is positioned across the lesion to be treated and expanded. The catheter is then withdrawn from the body.

In the physiological environment, the therapeutic agent molecules migrate out of the coating and deliver a therapeutically effective amount of the therapeutic agent at the treatment site. A defined amount of therapeutic agent remains locked-in the coating. The coating begins to degrade and results in the degradation-associated release of the therapeutic agent, as indicated in Block 608. The amount of therapeutic agent released is sufficient to provide a therapeutically effective amount of therapeutic agent as a result of degradation of the coating, as indicated in Block 610. In one embodiment of the invention, the therapeutic agent reduces inflammation at the treatment site.

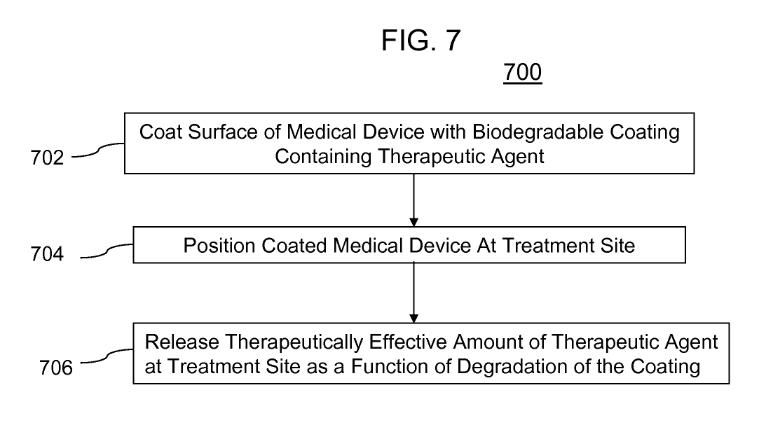

FIG. 7 is a flowchart of method 700 for improving the performance of a medical device. The method comprises coating the surface of a medical device with a biodegradable coating including, within the coating, a therapeutic agent, as shown in Block 702.

Next, as shown in Block 704, the coated device is placed at a treatment site. Finally, as shown in Block 706, a therapeutically effective amount of the therapeutic agent is released at the treatment site as a function of the degradation of the coating. In one embodiment of the invention, the therapeutic agent reduces inflammation at the treatment site, and thereby improves the performance of the medical device.

While the invention has been described with reference to particular embodiments, it will be understood by one skilled in the art that variations and modifications may be made in form and detail without departing from the spirit and scope of the invention.

1. A system for treating a vascular condition comprising:
a catheter;
a stent disposed on the catheter,
a coating disposed on the surface of the stent; and
at least one therapeutic agent within the coating, wherein the coating retains a predetermined amount of therapeutic agent that is sufficient to allow release of a therapeutically effective amount of therapeutic agent as a function of degradation of the coating.

2. The system of claim 1 wherein the coating comprises at least one polymer selected from the group consisting of polylactic acid, polyglycolic acid, polylactic-co-glycolic acid, caproic acid, polyethylene glycol, poly-trimethylene carbonate, polyanhydrides, polyacetates, polycaprolactones, poly(orthoesters), polyamides, polyurethanes, poly-alpha hydroxyesters and other suitable polymers.

3. The system of claim 1 wherein the at least one therapeutic agent is selected from the group consisting of anticoagulants, antiinflammatories, fibrinolytics, antiproliferatives, antibiotics, therapeutic proteins, recombinant DNA products, bioactive agents, diagnostic agents, radioactive isotopes, and radiopaque substances.

4. The system of claim 1 wherein the therapeutic agent carried by the coating reduces inflammation at the treatment site.

5. The system of claim 1 wherein the therapeutic agent is selected from the group consisting of zotarolimus, everolimus, sirolimus, pimecrolimus, dexamethasone, hydrocortisone, salicylic acid, fluocinolone acetonide, corticosteroids, prodrugs thereof, and combinations thereof.

6. The system of claim 5 wherein the coating comprises polylactic-co-glycolic acid.

7. A stent having a coating disposed on at least a portion of the surface of the stent and at least one therapeutic agent within the coating, wherein the coating retains a predetermined amount of therapeutic agent that is sufficient to allow release of a therapeutically effective amount of therapeutic agent as a function of degradation of the coating.

8. The stent of claim 7 wherein the coating comprises at least one polymer selected from the group consisting of polylactic acid, polyglycolic acid, polylactic-co-glycolic acid, caproic acid, polyethylene glycol, poly-trimethylene carbonate, polyanhydrides, polyacetates, polycaprolactones, poly(orthoesters), polyamides, polyurethanes, poly-alpha hydroxyesters and other suitable polymers.

9. The stent of claim 7 wherein the at least one therapeutic agent is selected from the group consisting of anticoagulants, antiinflammatories, fibrinolytics, antiprolifratives, antibiotics, therapeutic proteins, recombinant DNA products, bioactive agents, diagnostic agents, radioactive isotopes, and radiopaque substances.

10. The stent of claim 7 wherein the therapeutic agent reduces inflammation at the treatment site.

11. The stent of claim 7 wherein the therapeutic agent is selected from the group consisting of zotarolimus, everolimus, sirolimus, pimecrolimus, dexamethasone, hydrocortisone, salicylic acid, fluocinolone acetonide, corticosteroids, prodrugs thereof, and combinations thereof.

12. The stent of claim 11 wherein the coating comprises polylactic-co-glycolic acid.

13. A method of treating a vascular condition comprising:
delivering a stent including a biodegradable coating and at least one therapeutic agent to a treatment site via catheter; and
delivering a therapeutically effective amount of the at least one therapeutic agent to the treatment site as a function of degradation of the coating.

14. The method of claim 13 further comprising selecting the coating to provide a desired rate of therapeutic agent delivery.

15. The method of claim 14 wherein the coating comprises polylactic-co-glycolic acid.

16. The method of claim 15 wherein the therapeutic agent is selected from the group consisting of zotarolimus, everolimus, sirolimus, pimecrolimus, dexamethasone, hydrocortisone, salicylic acid, fluocinolone acetonide, corticosteroids, prodrugs thereof, and combinations thereof.

17. The method of claim 13 further comprising reducing inflammation at the treatment site.

18. A method of improving a performance of a medical device comprising:
providing at a treatment site a medical device having a biodegradable coating disposed on a surface of the device;
including within the coating at least one therapeutic agent; and
releasing a therapeutically effective amount of the therapeutic agent at the treatment site at a predetermined rate as a function of degradation of the coating.

19. The method of claim 18 further comprising reducing inflammation at the treatment site.
